(19) United States
(12) Patent Application Publication
(10) Pub. No.: US 2006/0212033 A1
(43) Pub. Date: Sep. 21, 2006

(54) Vertebral stabilization using flexible rods

(75) Inventors: Richard H. Rothman, Philadelphia, PA (US); Rafail Zubok, Midland Park, NJ (US); Mikhail Kvitnitsky, Clifton, NJ (US)

Correspondence Address: KAPLAN GILMAN GIBSON & DERNIER L.L.P., 900 ROUTE 9 NORTH, WOODBRIDGE, NJ, 07095, US

(73) Assignee: Accin Corporation

(21) Appl. No.: 11/186,699

(22) Filed: Jul. 21, 2005

Related U.S. Application Data
(60) Provisional application No. 60/658,365, filed on Mar. 3, 2005.

(51) Int. Cl. A61B 17/70

(52) U.S. Cl. 606/61; 606/72

(57) ABSTRACT

A stabilization rod for implantation in a patient, comprising: an elongate body including first and second ends; a flexible section disposed between the first and second ends, and including a first bore within the body and at least one through cut extending helically about the first bore to form a spring; and first and second fastening zones located at the first and second ends, respectively, and each operable to couple to a bone anchor.

### CROSS REFERENCE TO RELATED APPLICATIONS

This application claims the benefit of U.S. Provisional Patent Application No.: 60/658,365, filed Mar. 3, 2005, the entire disclosure of which is hereby incorporated by reference.

### BACKGROUND OF THE INVENTION

The present invention is directed to vertebral stabilization of a spine using flexible interconnecting rods.

Back pain is one of the most common and often debilitating conditions affecting millions of people in all walks of life. Today, it is estimated that over ten million people in the United States alone suffer from persistent back pain. Approximately half of those suffering from persistent back pain are afflicted with chronic disabling pain, which seriously compromises a person's quality of life and is the second most common cause of worker absenteeism. Further, the cost of treating chronic back pain is very high, even though the majority of sufferers do not receive treatment due to health risks, limited treatment options and inadequate therapeutic results. Thus, chronic back pain has a significantly adverse effect on a person's quality of life, on industrial productivity, and on heath care expenditures.

Degenerative spinal column diseases, such as disc degenerative diseases (DDD), spinal stenosis, spondylolisthesis, and so on, need surgical operation if they do not take a turn for the better by conservative management. Typically, spinal decompression is the first surgical procedure that is performed. The primary purpose of decompression is to reduce pressure in the spinal canal and on nerve roots located therein by removing a certain tissue of the spinal column to reduce or eliminate the pressure and pain caused by the pressure. If the tissue of the spinal column is removed the pain is reduced but the spinal column is weakened. Therefore, fusion surgery (e.g., ALIF, PLIF or posterolateral fusion) is often necessary for spinal stability following the decompression procedure. However, following the surgical procedure, fusion takes additional time to achieve maximum stability and a spinal fixation device is typically used to support the spinal column until a desired level of fusion is achieved. Depending on a patient's particular circumstances and condition, a spinal fixation surgery can sometimes be performed immediately following decompression, without performing the fusion procedure. The fixation surgery is performed in most cases because it provides immediate postoperative stability and, if fusion surgery has also been performed, it provides support of the spine until sufficient fusion and stability has been achieved.

Conventional methods of spinal fixation utilize a rigid spinal fixation device to support an injured spinal part and prevent movement of the injured part. These conventional spinal fixation devices include: fixing screws configured to be inserted into the spinal pedicle or sacral of the backbone to a predetermined depth and angle, rods or plates configured to be positioned adjacent to the injured spinal part, and coupling elements for connecting and coupling the rods or plates to the fixing screws such that the injured spinal part is supported and held in a relatively fixed position by the rods or plates.

U.S. Pat. No. 6,193,720 discloses a conventional spinal fixation device, in which connection members of a rod or plate type are mounted on the upper ends of at least one or more screws inserted into the spinal pedicle or sacral of the backbone. The connection units, such as the rods and plates, are used to stabilize the injured part of the spinal column which has been weakened by decompression. The connection units also prevent further pain and injury to the patient by substantially restraining the movement of the spinal column. However, because the connection units prevent normal movement of the spinal column, after prolonged use, the spinal fixation device can cause ill effects, such as “junctional syndrome” (transitional syndrome) or “fusion disease” resulting in further complications and abnormalities associated with the spinal column. In particular, due to the high rigidity of the rods or plates used in conventional fixation devices, the patient's fixed joints are not allowed to move after the surgical operation, and the movement of the spinal joints located above or under the operated area is increased. Consequently, such spinal fixation devices cause decreased mobility of the patient and increased stress and instability to the spinal column joints adjacent to the operated area.

It has been reported that excessive rigid spinal fixation is not helpful to the fusion process due to load shielding caused by rigid fixation. Thus, trials using load sharing semi-rigid spinal fixation devices have been performed to eliminate this problem and assist the bone fusion process. For example, U.S. Pat. Nos. 5,672,175; and 5,540,688; and U.S. Patent Publication No. 2001/0037111 disclose dynamic spine stabilization devices having flexible designs that permit axial load translation (i.e., along the vertical axis of the spine) for bone fusion promotion. However, because these devices are intended for use following a bone fusion procedure, they are not well-suited for spinal fixation without fusion. Thus, in the end result, these devices do not prevent the problem of rigid fixation resulting from fusion.

To solve the above-described problems associated with rigid fixation, non-fusion technologies have been developed. The Graf band is one example of a non-fusion fixation device that is applied after decompression without bone fusion. The Graf band is composed of a polyethylene band and pedicle screws to couple, the polyethylene band to the spinal vertebrae requiring stabilization. The primary purpose of the Graf band is to prevent sagittal rotation (flexion instability) of the injured spinal parts. Thus, it is effective in selected cases but is not appropriate for cases that require greater stability and fixation. See, Kanayama et al, Journal of Neurosurgery 95(1 Suppl):5-10, 2001, Markwalder & Wenger, Acta Neurochrgica 145(3):209-14.). Another non-fusion fixation device called “Dynesys” has recently been introduced. See Stoll et al, European Spine Journal 11 Suppl 2:S170-8, 2002, Schmoelz et al, J of spinal disorder & techniques 16(4):418-23, 2003. The Dynesys device is similar to the Graf band except it uses a polycarburethane spacer between the screws to maintain the distance between the heads of two corresponding pedicle screws and, hence, adjacent vertebrae in which the screws are fixed. Early reports by the inventors of the Dynesys device indicate it has been successful in many cases. However, it has not yet been determined whether the Dynesys device can maintain long-term stability with flexibility and durability in a controlled study. Because it has polyethylene components and interfaces, there is a risk of mechanical failure. Furthermore, due to the mechanical configuration of the device, the surgical technique required to attach the device to the spinal column is complex and complicated.

U.S. Pat. Nos. 5,282,863 and 4,748,260 disclose a flexible spinal stabilization system and method using a plastic, non-metallic rod. U.S. patent publication No. 2003/0083657 discloses another example of a flexible spinal stabilization device that uses a flexible elongate member. These devices are flexible but they are not well-suited for enduring long-term axial loading and stress. Additionally, the degree of desired flexibility vs. rigidity may vary from patient to patient. The design of existing flexible fixation devices are not well suited to provide varying levels of flexibility to provide optimum results for each individual candidate. For example, U.S. Pat. No. 5,672,175 discloses a flexible spinal fixation device which utilizes a flexible rod made of metal alloy and/or a composite material. Additionally, compression or extension springs are coiled around the rod for the purpose of providing de-rotation forces on the vertebrae in a desired direction. However, this patent is primarily concerned with providing a spinal fixation device that permits “relative longitudinal translational sliding movement along [the] vertical axis” of the spine and neither teaches nor suggests any particular designs of connection units (e.g., rods or plates) that can provide various flexibility characteristics. Prior flexible rods such as that mentioned in U.S. Pat. No. 5,672,175 typically have solid construction with a relatively small diameter in order to provide a desired level of flexibility. Because they are typically very thin to provide suitable flexibility, such prior art rods are prone to mechanical failure and have been known to break after implantation in patients.

U.S. Patent Publication No. 2005/0065516 discloses a flexible spinal fixation device having a flexible rod for non-rigid stabilization of the spinal column. In one embodiment, the fixation device includes at least two securing members configured to be inserted into respective adjacent spinal pedicles, each securing member each including a coupling assembly. The fixation device further includes a flexible rod configured to be received and secured within the coupling assemblies of each securing member so as to flexibly stabilize the affected area of the spine. Among the problems with the flexible rod of U.S. Patent Application No. 20050065516 is that there appears to be little or no compressive movement of the rod, which is a characteristic of conventional solid rods.

While U.S. Pat. No. 6,835,205 discloses use of flexible springs instead of rods for stabilizing adjacent vertebrae of a spine, the springs do not possess the physical characteristics of a rod, for example, in the way they are connected to pedicle screws.

Therefore, conventional spinal fixation devices have not provided a comprehensive and balanced solution to the problems associated with curing spinal diseases. Many of the prior devices are characterized by excessive rigidity, which leads to the problems discussed above while others, though providing some flexibility, are not well-adapted to provide varying degrees of flexibility. Additionally, existing flexible fixation devices utilize non-metallic components that are not proven to provide long-term stability and durability.

### SUMMARY OF THE INVENTION

In accordance with one or more embodiments of the present invention, a stabilization rod for implantation in a patient includes: an elongate body including first and second ends; a flexible section disposed between the first and second ends, and including a first bore within the body and at least one through cut extending helically about the first bore to form a spring; and first and second fastening zones located at the first and second ends, respectively, and each operable to couple to a bone anchor. The flexible section may be formed from one continuous through cut extending helically about the first bore.

In accordance with one or more embodiments of the present invention, a system for stabilizing a plurality of bones of a patient includes: a first stabilization rod including an elongate body having first and second ends, and a flexible section disposed between the first and second ends, the flexible section including a first bore within the body and at least one through cut extending helically about the first bore to form a spring; a first bone anchor operable to fixedly connect to one of the bones of the patient, and including a first coupling element at one end thereof operable to couple to the first end of the first stabilization rod; and a second bone anchor operable to fixedly connect to another of the bones of the patient, and including a second coupling element at one end thereof operable to couple to the second end of the first stabilization rod.

The system may further include: a second stabilization rod including an elongate body having first and second ends; and a third bone anchor operable to fixedly connect to another of the bones of the patient, and including a first coupling element at one end thereof operable to couple to the second end of the second stabilization rod. The first and second stabilization rods may include respective coupling elements operable to engage one another at the second bone anchor. Preferably, the coupling of the first stabilization rod includes a post extending longitudinally from the elongate body, the post having one or more cross-sectional dimensions smaller than those of the elongate body. The coupling of the second stabilization rod may include a bore extending longitudinally within the elongate body and at least one slot extending from the bore to a surface of the body. Preferably, the post and the bore are sized and shaped to engage one another. Activation of the coupling element of the second bone anchor preferably applies compressive force on the coupling of the second stabilization rod and causes an internal surface of the bore thereof to engage the post, thereby fastening the first and second stabilization rods together.

The system may further include: one or more further stabilization rods in operative connection to at least one of the first and second stabilization rods; and one or more further bone anchors in operative engagement with the further stabilization rods.

Other aspects, features, advantages, etc. will become apparent to one skilled in the art when the description of the preferred embodiments of the invention herein is taken in conjunction with the accompanying drawings.

### BRIEF DESCRIPTION OF THE DRAWINGS

For the purposes of illustrating the various aspects of the invention, there are shown in the drawings forms that are presently preferred, it being understood, however, that the invention is not limited to the precise arrangements and instrumentalities shown.

FIGS. 1A-1B illustrate perspective and side (or lateral) views, respectively, of an intervertebral stabilizer in accordance with one or more embodiments of the present invention;

FIGS. 2A-2B illustrate posterior and side (or lateral) views, respectively, of the intervertebral stabilizer of FIGS. 1A-1B in use in accordance with one or more embodiments of the present invention;

FIGS. 3A-3D illustrate perspective views (FIG. 3B being a cross-sectional view of FIG. 3A) of a stabilization rod of the intervertebral stabilizer of FIGS. 1A-1B or one or more further embodiments herein;

FIG. 4 illustrates a perspective view of a multilevel intervertebral stabilizer in accordance with one or more embodiments of the present invention;

FIG. 5 illustrates a set of stabilization rods that may be used with the intervertebral stabilizer of one or more of the embodiments herein;

FIGS. 6A-6C illustrate perspective views of further alternative embodiments of a stabilization rod in accordance with one or more embodiments of the present invention;

FIGS. 7A-7B illustrate perspective views of a further alternative embodiment of a stabilization rod in accordance with one or more embodiments of the present invention.

### DETAILED DESCRIPTION

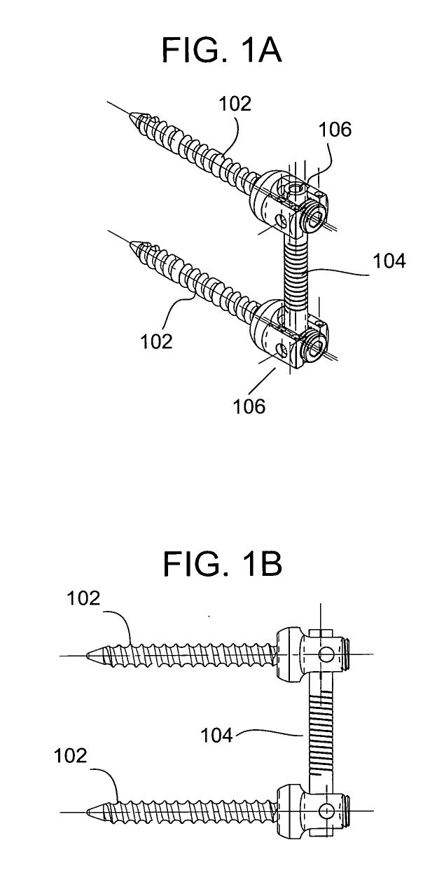

FIGS. 1A-B illustrate an embodiment of a spinal stabilizer 100 in accordance with one or more aspects of the present invention. This embodiment of the stabilizer 100 is designed for single level spinal stabilization, preferably from the posterior of the spine. It is understood that the use of the various embodiments of the invention discussed herein has been directed to a specific application of stabilizing the spine; however, other applications are contemplated without departing from the scope of the invention. Indeed, any application in which a conventional solid or flexible rod is called for to stabilize two anatomical bodies is within the scope of the invention.

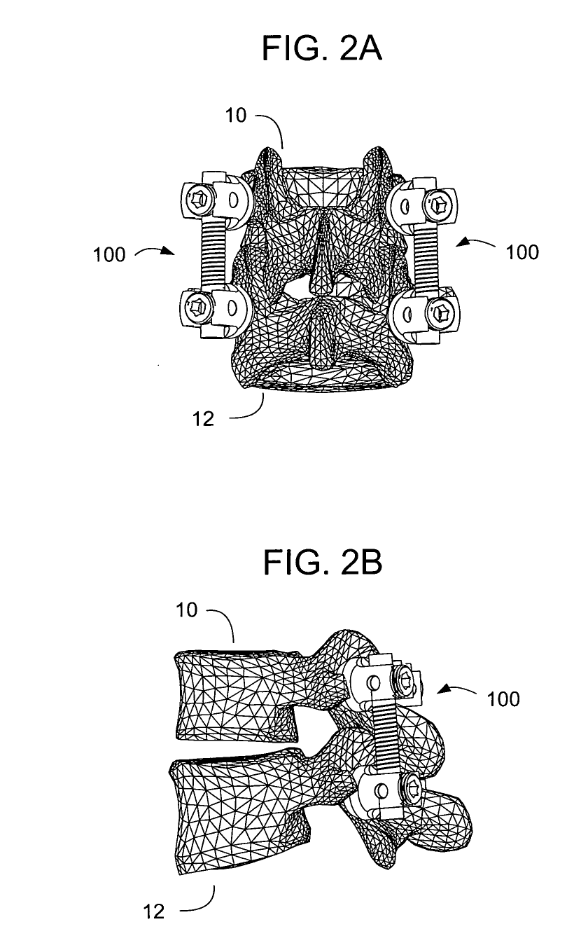

As best seen in FIGS. 2A-B, the stabilizer 100 is sized and shaped for bilateral use on a posterior aspect of the spine. In particular, the stabilizer 100 provides stabilization with respect to adjacent vertebral bones 10, 12 of the spine. It is understood that the size and shape of the stabilizer 100 may be adapted to fit at any level of the spine, such as the cervical spine, thoracic spine, or lumbar spine. The posterior stabilizer 100 is preferably used at the early to moderate stages of the spinal disc degeneration disease process to inhibit posterior disc, vertebral foramen, and inferior vertebral notch collapse with the minimal (semi-constrained) restriction of the vertebral body biological range of motion.

The stabilizer 100 includes first and second anchoring elements 102, such as pedicle screws, and a flexible rod 104 coupled to the screws 102. It is understood that the anchoring elements need not be pedicle screws; indeed, any of the known techniques of coupling a conventional rod (e.g., a solid rod) may be employed without departing from the invention. For example, posts may be used. It is contemplated that the stabilizer 100 may employ any pedicle screw system presently utilizing a solid fixation rod of any diameter. In this regard, the depicted embodiment employs an articulating tulip 106 coupled to each screw 102 that may be moved into various positions with respect to the screw 102. The tulips 106 are adapted to receive and clamp respective ends of the flexible rod 104.

In use to stabilize a portion of a spine, the anchors 102 secure the respective ends of the flexible rod 104 to respective portions of two adjacent vertebrae 10, 12 in order to inhibit posterior disc collapse with minimum restriction of the inter-vertebral movement. For example, the respective screws 102 may be fixed to a respective articular process of each adjacent vertebrae 10, 12, a respective transverse process of each adjacent vertebrae, a respective pedicle of each adjacent vertebrae, or other suitable respective portions of the adjacent vertebrae 10, 12.

It is most preferred that the system is disposed bilaterally, where one dynamic stabilizer 100 is disposed on each side of the spinous process and attached to respective adjacent vertebrae in a posterior location as discussed above. As will be discussed in further detail below, the flexible rod 104 preferably does not permit compression beyond a certain point at which the respective coils of the spring are in contact with one another. Thus, the dynamic stabilizers 100 located on each side of the spinous process inhibit posterior disc collapse. As the flexible rod 104 provides extension when tensile forces are applied, the dynamic stabilizer does not substantially limit displacement, rotation, subluxation, flexion, extension, bending, or any combination thereof as between the adjacent vertebrae.

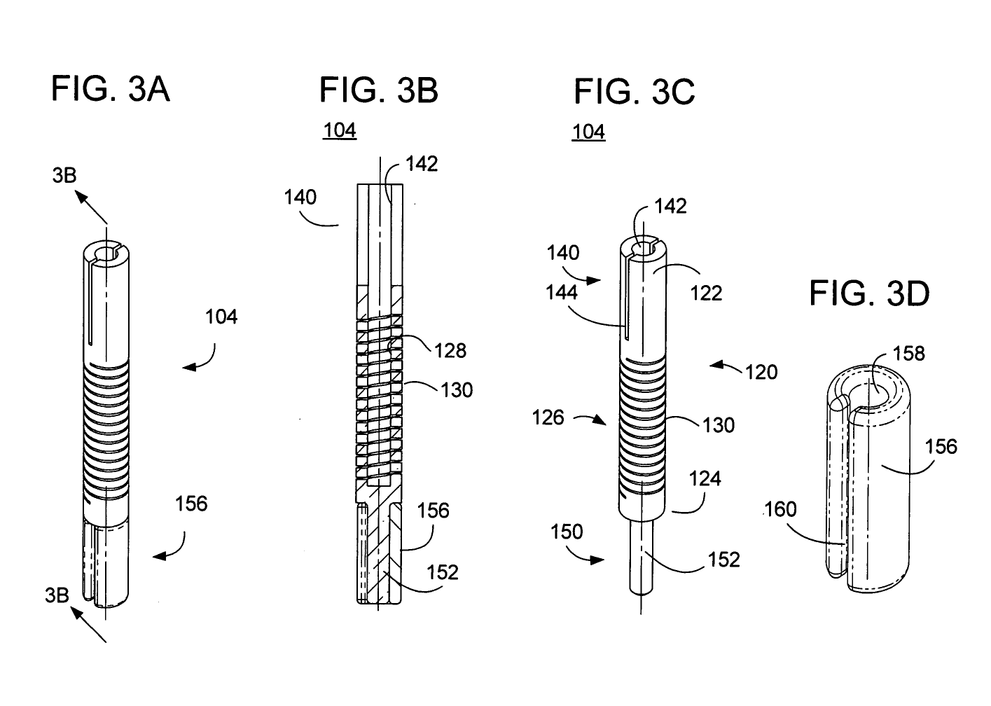

With reference to FIGS. 3A-D, the flexible rod (or stabilization rod) 104 includes an elongate body 120 having first and second ends 122, 124, respectively. A flexible section 126 is preferably disposed between the first and second ends 122, 124. As best seen in FIG. 1A, the first and second ends 122, 124 are utilized as fastening zones for coupling to the respective bone anchors 102. The flexible section 126 includes a first bore 128 within the body 120 and at least one through-cut 130 extending helically about and into the first bore 128 to form a spring. Preferably, the flexible section 126 is formed from one continuous through-cut 130 extending helically along the body 120.

Although the cross-section of the rod 104 may take on any shape, such as rectangular, square, triangular, hexagonal, octagonal, polygonal, or any combination thereof. It is preferred that the cross-section is round and the rod 104 is of a generally cylindrical configuration. In a preferred embodiment, the diameter of the flexible rod 104 is preferably about 5.5 mm such that widely available bone anchors 102 and tulips 106 may be employed to engage the rod 104.

A coupling element is preferably disposed at at least one of the first end 122 and the second end 124 of the body 120, where each coupling is preferably operable to connect to a further stabilization rod, such as a rod of similar characteristics as the rod 104. For example, the first end 122 may include a coupling 140 having a second bore 142 extending longitudinally within the body 120 and at least one slot 144 extending from the second bore 142 to a surface of the body 120. Preferably, the first bore 128 and the second bore 142 are respective portions of one continuous bore within the elongate body 120. As best seen in FIG. 1A, the first end 122 of the body 120 is of a diameter suitable for reception in the tulip 106 of one of the bone anchors 102.

By way of further example, the second end 124 of the body 120 may include a coupling 150 having a post 152 extending longitudinally from the elongate body 120. Preferably, the post 152 has one or more cross-sectional dimensions smaller than the cross-sectional dimension of the elongate body 120. In one or more embodiments, the post 152 may be received in the tulip 106; however, given that the diameter of the post 152 is generally smaller than the diameter of the body 120, the tulip 106 may require a specialized configuration to engage the post 152. Accordingly, further embodiments of the present invention preferably contemplate the provision of a sleeve 156 having a bore 158 that is sized and shaped to receive the post 152. The sleeve 156 preferably includes at least one slot 160 extending from the bore 158 to a surface of the sleeve 156 and also extending the length of the sleeve 156. Thus, the sleeve is preferably operable to clamp around the post 152 and to compliment the cross-sectional dimension of the post 152 to match the diameter of the elongate body 120. Preferably, a length of the sleeve 156 is substantially the same length as the post 152. Consequently, the second end 124 (including the sleeve 156) may be received in the tulip 106 of the bone anchor 102 without requiring specialized dimensioning of the tulip 106. The activation of the tulip 106 to clamp the second end 124 of the rod 104 preferably applies a compression force on the sleeve 156 and causes an internal surface of the bore 158 to engage the post 152.

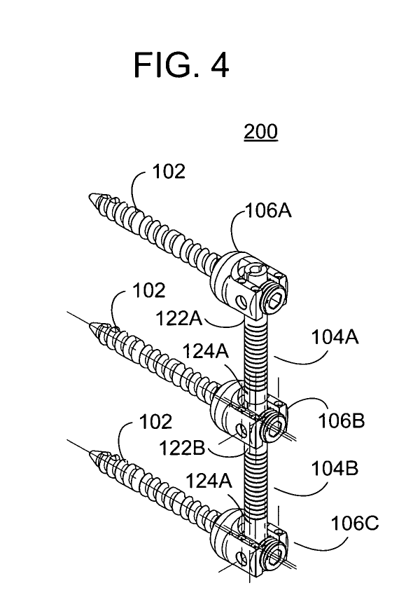

With reference to FIG. 4, one or more embodiments of the present invention contemplate multilevel spinal stabilization, again from the posterior of the spine. The multilevel stabilizer 200 of FIG. 4 includes three bone anchors 102, each with a respective tulip 106 and two stabilization rods 104A, 104B that are coupled together at the central tulip 106. This embodiment therefore accommodates two level stabilization. A first end 122A of the stabilization rod 104A is coupled to the bone anchor 102 by way of the tulip 106A. A second end 124B of the stabilization rod 104B (e.g., including a respective sleeve 156) is preferably connected to the lower tulip 106C. The stabilization rods 104A, 104B are preferably coupled together at the tulip 106B, as will be discussed in more detail below.

Turning again to FIGS. 3A-D, the bore 142 and the slot 144 of the stabilization rod 104 preferably operate such that a compressive force about the first end 122 of the body 120 causes a diameter of the second bore 142 to reduce. By way of example, the stabilization rods 104A, 104B may include couplings 140, 150 of substantially similar configuration. In such a scenario, the bore 142 of stabilization rod 104B and the post 152 of the stabilization rod 104A may be sized and shaped such that the post 152 of the stabilization rod 104A may be received into the bore 142 of the stabilization rod 104B. Compression of the first end 122B of the stabilization rod 104A preferably causes an internal surface of the second bore 142 to engage the post 152 of the stabilization rod 104A, thereby fastening the stabilization rods 104A, 104B together. In a preferred embodiment, the tulip 106B of the bone anchor 102 preferably provides the compression force on the first end 122B of the stabilization rod 104B when the rod 104B is secured to the tulip 106C.

Consequently, the stabilizer 200 provides multilevel stabilization by cascading respective stabilization rods 104. Those skilled in the art will appreciate that the multilevel stabilizer 200 may include further stages, not shown, to accommodate yet further levels of stabilization.

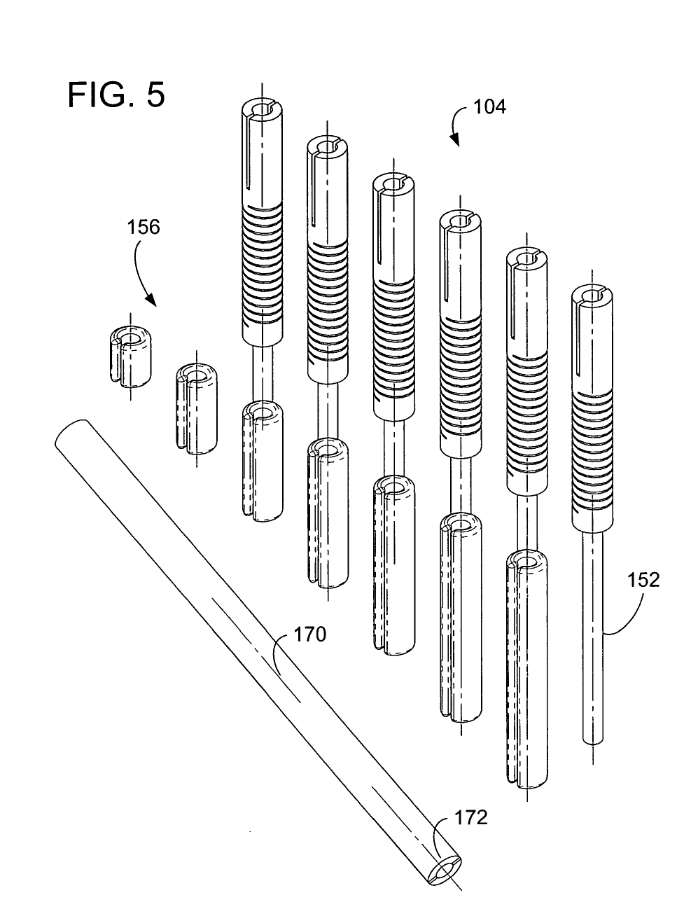

With reference to FIG. 5, the surgeon is preferably provided with a plurality of stabilization rods 104, one or more of which exhibits a different physical property. For example, each of the rods 104 may include a post 152 of different length and a correspondingly sized sleeve 156. For example, the collection of rods 104 may exhibit lengths of about 45 mm to about 75 mm in, for example, 5 mm increments. The incremental lengths may be achieved by way of the varying lengths of the posts 152 and corresponding sleeves 156. In a preferred embodiment, the differing lengths of the rods 104 and sleeves 156 are color coded for ease of identification during the surgical procedure.

Further, the collection of rods may include a solid rod 170 including a slotted bore at at least one end 172 thereof. Additional variations in the lengths of the rods 104 may be achieved by cutting off a section of the post 152 and/or sleeve 156, or by cutting off a section of the solid rod 170.

Advantageously, the collection of stabilization rods 104 provides an inventory to the surgeon from which to vary the length and flexibility regions of the rod(s) 104 and to accommodate differing anatomical conditions.

Turning again to FIG. 4, it is noted that a stabilization rod 104 having a relatively long post 152 and sleeve 156 may be employed in the multilevel stabilizer 200 of FIG. 5. Indeed, a single rod 104 (including the sleeve 156) may provide flexibility at one level and no flexibility at the other level. Indeed, the post 152 and sleeve 156 portion of the stabilization rod 104 may span between the central tulip 106B and the lower tulip 106C, while the flexible section 126 of the rod 104 may span from the central tulip 106B to the upper tulip 106A. The central tulip 106B and the lower tulip 106C may provide compression force to clamp the sleeve 156 about the post 152. The resulting multilevel stabilizer 200 would include an upper flexible portion of the rod 104 and a lower rigid portion of the same rod 104. A variant of the above could include the flexible rod 104 and the rigid rod 170.

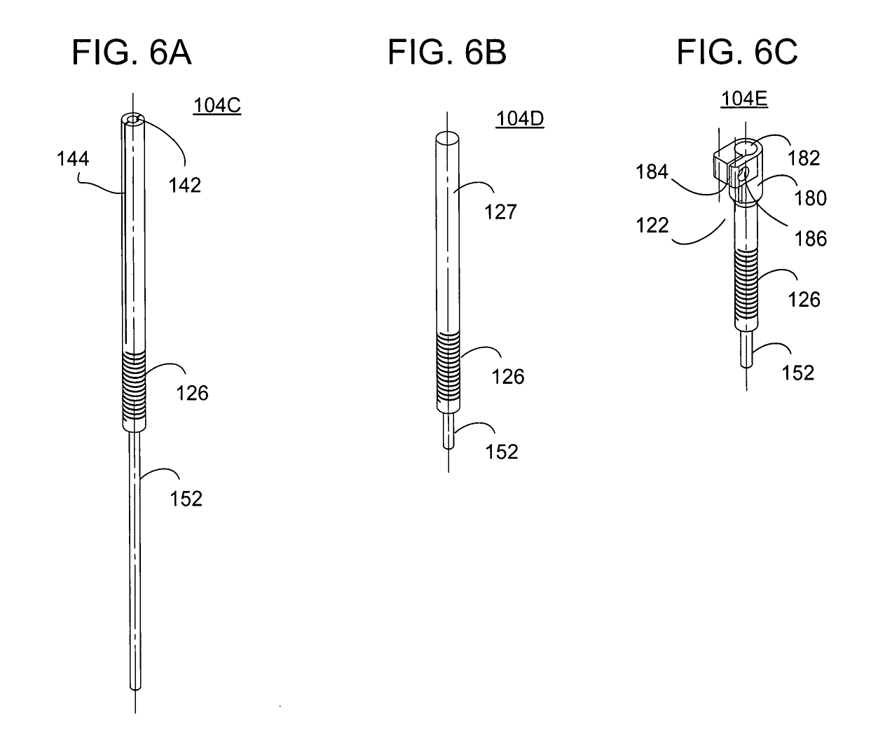

With reference to FIG. 6A, a further embodiment of a stabilization rod 104C is illustrated in which a length of the slotted bore 142, 144 is relatively longer than a length of the flexible element 126. Alternatively, or in addition, a length of the post 152 and/or the sleeve 156 (not shown) is relatively longer than the length of the flexible element 126. Note that this configuration is in contrast to that of FIG. 3C, where a length of the flexible element 126 is relatively longer than a length of the slotted bore 142, 144 and relatively longer than a length of the post 152 and/or sleeve 156. In a preferred embodiment, the length of the stabilization rod 104C may be customized by the surgeon by cutting off a section of the slotted bore 142, 144 and/or cutting off a section of the post 152 and/or sleeve 156. Advantageously, the stabilization rod 104C reduces inventory while providing significant flexibility in terms of varying a length of the rod 104C to accommodate differing anatomical conditions. It is noted that the stabilization rod 104C may be employed in single level stabilization and/or multilevel stabilization. The stabilization rod 104D may be coupled to one or more further stabilization rods 104 or 170 (or other embodiments herein) by way of the slotted bore 142, 144 and/or post 152 as has been discussed in detail in the embodiments above.

With reference to FIG. 6B, a further embodiment of a stabilization rod 104D is illustrated. The stabilization rod 104D preferably includes a substantially rigid section 127 instead of the slotted bore 142, 144 of FIG. 6A. In one or more embodiments, a length of the post 152 is relatively shorter than the length of the flexible section 126. In alternative embodiments, the length of the post 152 may be substantially longer than a length of the flexible section 126. Advantageously, a length of the stabilization rod 104D may be adjusted by cutting off a section of the rigid section 127 and using a terminal end thereof as the first end 122 of the elongate body 120.

As will be apparent to one skilled in the art from the description herein, the stabilization rod 104D may be employed in single or multilevel stabilization applications. A sleeve 156 may be employed to compliment the diameter of the post 152 such that respective ends of the stabilization rod 104D may be coupled to respective bone anchors 102. In a multilevel application, the stabilization rod 104D may be used to provide one level of flexibility and another level of rigid stabilization as has been discussed hereinabove with respect to the stabilization rods 104 of FIG. 5. Alternatively, the stabilization rod 104D may be coupled to one or more further stabilization rods 104 or 170 (or other embodiments) by way of the post 152 as has been discussed in detail in the embodiments above.

With reference to FIG. 6C, a further embodiment of a stabilization rod 104E is illustrated. The stabilization rod 104E preferably includes a coupling 180 disposed at the first end 122 of the body 102, which is operable to connect to a further stabilization rod 104, 170, or other embodiment. The coupling 180 is preferably integral with the rod 104E. The coupling 180 preferably includes a bore 182 extending longitudinally away from the elongate body 102, and at least one slot 184 extending from the bore 182 to a surface of the coupling 180. The bore 182 is preferably sized and shaped to receive an end of a further stabilization rod. As illustrated, unlike the bore 142, the diameter of the bore 182 is preferably larger, such as to permit connection to the diameter of the elongate body 102. By way of example, the bore 182 of the stabilization rod 104E is preferably capable of receiving an end of the rigid rod 170, the sleeve 156, the rigid portion 127 (FIG. 6B), etc.

Tightening means 186 is preferably operable to cause a diameter of the bore 182 to reduce in response to actuation of the tightening means 186. Actuation of the tightening means 186 preferably causes an internal surface of the bore 182 to engage the end of the further stabilization rod, thereby fastening the rods together. By way of example, the tightening means 186 may be implemented as a cylindrical clamp, including first and second flanges disposed on opposing sides of the slot 184, and at least one threaded element operable (e.g., a screw) operable to draw the flanges together when turned into a threaded portion of the one of the flanges.

Notably, the stabilization rod 104E may alternatively or in addition include a post 152 of relatively short length as illustrated or of relatively longer length as in other embodiments discussed herein.

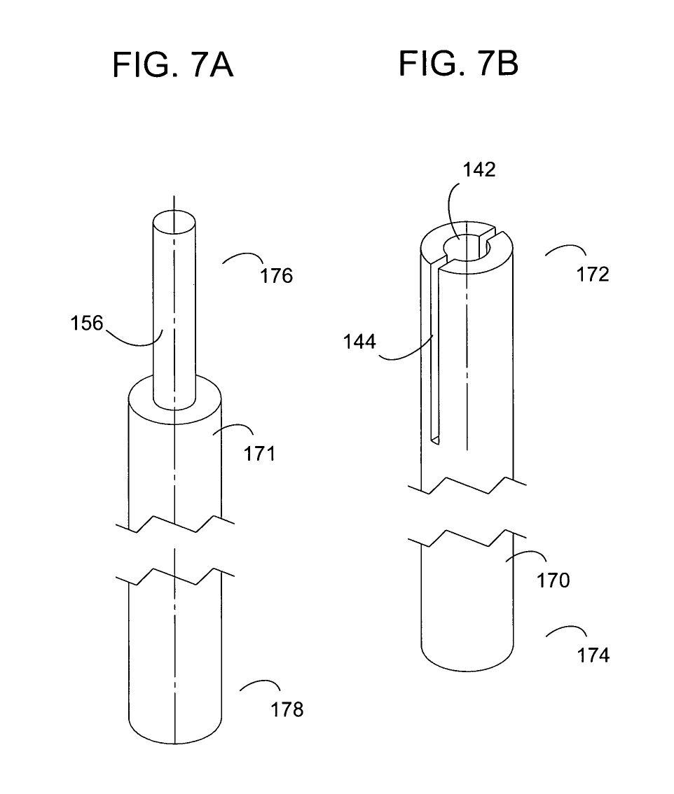

With reference to FIGS. 7A-B, a stabilization rod system may include a first elongate body 170 having first and second ends 172, 174. The first end 172 preferably includes a slotted bore 142, 144 as has been discussed in connection with previous embodiments. The system also includes a second elongate body 171 having first and second ends 176, 178. The first and second bodies 170, 171 are preferably rigid, although further embodiments may include one or more flexible portions. The first end 176 of the second body 171 preferably includes a post 152 as has been discussed in detail with respect to other embodiments herein. The bore 142 and post 152 are preferably sized and shaped to couple with one another such that compressive force about the first end 172 of the first body 170 causes a diameter of the bore 142 to reduce and engage the post 152.

The stabilization rod system of FIGS. 7A-B are preferably employed for multilevel stabilization in which a respective tulip 106 of the bone anchor 102 provides the compressive force on the slotted bore 142, 144 to secure the bodies 170, 171 together. A surgeon may customize a length of the stabilization rod by cutting off a portion of the first body 170 and/or the second body 171. The terminal ends 174, 178 of the bodies 170, 171, respectively, are preferably coupled to bone anchors 102 by way of respective tulips 106 as has been discussed in detail hereinabove.

Preferably the components discussed above are formed from CP Titanium or Titanium Alloy, Stainless Steel, Cobalt Chromium Alloy, Plastics and/or other biologically acceptable materials.

The portions of the device may be produced in the range of sizes and length adequate to the requirements.

Some notable features of the aforementioned stabilizers are listed below, it being understood that various features alone or in combination may be employed:

posterior disc collapse inhibited with the minimal restriction of the vertebral body biological ROM;

minimum pre-determined distance between bone anchors (or any attachment points) maintained without limiting displacement, rotation, subluxation, flexion, extension, bending or any combination thereof;

locking mechanism accommodating any existing screw system presently used for solid rod fixation;

hybrid multilevel system configurations permitted;

controlled system flexibility permitted;

cross-section can be circular, square, rectangular, polygonal and any combination thereof; and

rigidity in specific direction controlled.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A stabilization rod for implantation in a patient, comprising:
an elongate body including first and second ends;
a flexible section disposed between the first and second ends, and including a first bore within the body and at least one through cut extending helically about the first bore to form a spring; and
first and second fastening zones located at the first and second ends, respectively, and each operable to couple to a bone anchor.

2. The stabilization rod of claim 1, wherein the flexible section is formed from one continuous through cut extending helically about the first bore.

3. The stabilization rod of claim 1, wherein the rod has a round cross-section.

4. The stabilization rod of claim 3, wherein a diameter of the body is substantially unchanged along a length thereof.

5. The stabilization rod of claim 1, further comprising a coupling disposed at the first end of the body and operable to connect to a further stabilization rod.

6. The stabilization rod of claim 5, wherein the coupling includes a second bore extending longitudinally within the elongate body and at least one slot extending from the second bore to a surface of the body such that a compressive force about the first end causes a diameter of the second bore to reduce.

7. The stabilization rod of claim 6, wherein the second bore is sized and shaped to receive a post from the further stabilization rod.

8. The stabilization rod of claim 7, wherein activation of a connector of the bone anchor to clamp the first fastening zone applies the compressive force and causes an internal surface of the second bore to engage the post of the further stabilization rod, thereby fastening the stabilization rods together.

9. The stabilization rod of claim 8, wherein the first and second bores are portions of one continuous bore within the elongate body.

10. The stabilization rod of claim 9, wherein a length of the flexible element is longer than a length of the slotted second bore.

11. The stabilization rod of claim 11, wherein a length of the slotted second bore is longer than a length of the flexible element.

12. The stabilization rod of claim 11, wherein an overall length of the stabilization rod is adjustable by cutting off a section of the slotted second bore.

13. The stabilization rod of claim 1, further comprising:
a coupling disposed at the first end of the body and operable to connect to a further stabilization rod, wherein:
the coupling includes a second bore extending longitudinally away from the elongate body, at least one slot extending from the second bore to a surface of the coupling, and tightening means operable to cause a diameter of the second bore to reduce; and
the second bore is sized and shaped to receive an end of a further stabilization rod.

14. The stabilization rod of claim 13, wherein a diameter of the second bore is substantially the same as a diameter of the elongate body.

15. The stabilization rod of claim 13, wherein activation of the tightening means causes an internal surface of the second bore to engage the end of the further stabilization rod, thereby fastening the stabilization rods together.

16. The stabilization rod of claim 13, wherein the tightening means includes first and second flanges disposed on opposing sides of the slot, and at least one threaded element operable to draw the flanges together when turned into at least one of the flanges.

17. The stabilization rod of claim 1, further comprising a coupling disposed at the second end of the body and operable to connect to a further stabilization rod.

18. The stabilization rod of claim 17, wherein the coupling includes a post extending longitudinally from the elongate body, the post having one or more cross-sectional dimensions smaller than those of the elongate body.

19. The stabilization rod of claim 18, wherein the post the second bore is sized and shaped for insertion into a mating bore of a further stabilization rod.

20. The stabilization rod of claim 19, further comprising a sleeve including a second bore sized and shaped to receive the post and at least one slot extending from the second bore to a surface of the sleeve such that a compressive force about the sleeve causes a diameter of the second bore to reduce.

21. The stabilization rod of claim 20, wherein the sleeve is sized and shaped to complement the one or more cross-sectional dimensions of the post to substantially match those of the elongate body.

22. The stabilization rod of claim 20, wherein a length of the sleeve is substantially the same as a length of the post.

23. The stabilization rod of claim 20, wherein activation of a connector of the bone anchor to clamp the second fastening zone applies the compressive force and causes an internal surface of the second bore to engage the post.

24. The stabilization rod of claim 18, wherein a length of the flexible element is longer than a length of the post and/or sleeve.

25. The stabilization rod of claim 18, wherein a length of the post and/or sleeve is longer than a length of the flexible element.

26. The stabilization rod of claim 25, wherein an overall length of the stabilization rod is adjustable by cutting off a section of the post and/or sleeve.

27. The stabilization rod of claim 1, further comprising a substantially rigid section of the body extending from the flexible element to the first end of the body.

28. The stabilization rod of claim 27, wherein at least one of: the rod has a round cross-section; and a diameter of the body is substantially unchanged along a length thereof.

29. The stabilization rod of claim 27, wherein a length of the flexible element is longer than a length of the rigid section.

30. The stabilization rod of claim 27, wherein a length of the rigid section is longer than a length of the flexible element.

31. The stabilization rod of claim 30, wherein an overall length of the stabilization rod is adjustable by cutting off a section of the rigid section and using a terminal end of the rigid section as the first end of the body.

32. A stabilization rod for implantation in a patient, comprising:
a first elongate body including first and second ends, the first end including a bore extending longitudinally within the elongate body and at least one slot extending from the bore to a surface of the first body; and
a second elongate body including first and second ends, the first end including a post extending longitudinally from the second body, the post being sized and shaped to insert into the bore of the first body.

33. The stabilization rod of claim 32 wherein compressive force about the first end of the first body causes a diameter of the bore to reduce and engage the post of the second elongate body.

34. A system for stabilizing a plurality of bones of a patient, comprising:
a first stabilization rod including an elongate body having first and second ends, and a flexible section disposed between the first and second ends, the flexible section including a first bore within the body and at least one through cut extending helically about the first bore to form a spring;
a first bone anchor operable to fixedly connect to one of the bones of the patient, and including a first coupling element at one end thereof operable to couple to the first end of the first stabilization rod; and
a second bone anchor operable to fixedly connect to another of the bones of the patient, and including a second coupling element at one end thereof operable to couple to the second end of the first stabilization rod.

35. The system of claim 34, further comprising:
a second stabilization rod including an elongate body having first and second ends; and
a third bone anchor operable to fixedly connect to another of the bones of the patient, and including a first coupling element at one end thereof operable to couple to the second end of the second stabilization rod,
wherein the first and second stabilization rods include respective coupling elements operable to engage one another at the second bone anchor.

36. The system of claim 35, wherein:
the coupling of the first stabilization rod includes a post extending longitudinally from the elongate body, the post having one or more cross-sectional dimensions smaller than those of the elongate body;
the coupling of the second stabilization rod includes a bore extending longitudinally within the elongate body and at least one slot extending from the bore to a surface of the body;
the post and the bore are sized and shaped to engage one another; and
activation of the coupling element of the second bone anchor applies compressive force on the coupling of the second stabilization rod and causes an internal surface of the bore thereof to engage the post, thereby fastening the first and second stabilization rods together.

37. The system of claim 36, further comprising:
one or more further stabilization rods in operative connection to at least one of the first and second stabilization rods; and
one or more further bone anchors in operative engagement with the further stabilization rods.

38. The system of claim 37, wherein the stabilization rods are in operative connection by way of respective bores and posts.
